(19) United States
(12) Patent Application Publication
(10) Pub. No.: US 2019/0019399 A1
(43) Pub. Date: Jan. 17, 2019

(54) DEVICES, PROCESSES AND COMPUTER PROGRAMS FOR AN ALARM SERVER, FOR AN ALARM SOURCE AND FOR AN ALARM GENERATOR, ALARM SYSTEM

(71) Applicant: Drägerwerk AG & Co. KGaA, Lubeck (DE)

(72) Inventors: Birger LANDWEHR, Lubeck (DE); Marco BERNUTZ, Stockelsdorf (DE)

(21) Appl. No.: 16/033,778

(22) Filed: Jul. 12, 2018

(30) Foreign Application Priority Data
Jul. 14, 2017 (DE) 10 2017 006 677.9

(51) Int. Cl. G08B 25/00; G16H 40/67; A61B 5/00; G08B 21/02

(52) U.S. Cl. CPC G08B 25/001; G08B 21/02; A61B 5/0022; G16H 40/67

(57) ABSTRACT

A device (10) for the alarm server (100) includes an interface (12) for communication with one or more alarm sources (200) and with one or more alarm generators (300). A memory (14) stores information relating to alarms. A control module (16) is configured to control the one or more interfaces (12) and the memory (14) and receive an indication relating to an alarm from an alarm source (200) via the one or more interfaces (12) and to store information relating to the alarm in the memory (14) and to receive an alarm request from an alarm generator (300) via the one or more interfaces and to provide information relating to the alarm for the alarm generator (300) upon receipt of the alarm request if information relating to the alarm is stored in the memory (14).

### CROSS REFERENCE TO RELATED APPLICATIONS

This application claims the benefit of priority under 35 U.S.C. § 119 of German Application 10 2017 006 677.9, filed Jul. 14, 2017, the entire contents of which are incorporated herein by reference.

### TECHNICAL FIELD

Exemplary embodiments pertain to devices, processes and computer programs for an alarm server, for an alarm source and for an alarm generator as well as to an alarm system, especially but not exclusively related to the communication of alarm information between an alarm source, an alarm server and an alarm generator in a distributed alarm system for primary alarm generation without availability monitoring of the recipients.

### BACKGROUND

Automated systems are increasingly used, for example, in hospitals for monitoring patients and detecting critical situations and correspondingly alarming/informing the clinical staff. Alarm systems are based on the detection and conveying of information on (information relating to) the state of alarm to selected recipients, who had registered in advance at the central alarm server. The alarm server continuously monitors the registered recipients with respect to their availability and it dynamically passes on the escalation paths of alarms. Since the knowledge about the possible recipients of an alarm message is stored, monitored and maintained at one location, this makes the alarm server a critical component. A redundant configuration of this component entails problems concerning synchronization, because the information is never present simultaneously everywhere and the effort for coordination and communication, which is necessary for the ability to ensure protection against failure, may lead to inconsistencies.

Alarms are conventionally passed on and escalated to recipients, whose availability status is known. For example, the document U.S. Pat. No. 7,439,856 B2 discloses a concept, in which the recipients of alarm messages are classified as first recipients and second recipients. First recipients are the recipients who shall receive the message primarily after a prior availability check, and it is only if these recipients are not available that the second recipients are notified, likewise after a prior availability check. If these recipients are likewise unavailable, a previously defined escalation process, which is not specified in more detail, will be run. The recipients of an alarm message are identified and their availability is checked in advance. A message is sent only in case of availability of a recipient and waiting times can thus be minimized. However, technical transmission risks, e.g., the breaking off of the connection or failure of the terminal, may be critical.

The document U.S. Pat. No. 8,842,001 B2 describes a process for continuously monitoring the connection between an alarm source and an alarm generator, which is responsible for the primary alarm generation. The process is based on the setting up of a dedicated heartbeat signal between the source and the recipient, after the recipient has logged in as a primary alarm generator at the alarm source, but still before a first alarm is transmitted. The recipient first registers as a primary alarm generator, before he can act as an alarm generator. This is, however, undesirable if the alarm generator shall act only as an additional second signaling unit. Registration as a primary alarm generator is not necessary in this case.

The document U.S. Pat. No. 8,912,897 B2 describes very generally (multi)parameter alarms with complex alarm detection logic. The detection logic analyzes messages, e.g., alarms, of the source system and decides which message will be forwarded to a web browser and will be displayed there. This solution utilizes a web browser. A selected message is displayed, but not interpreted or converted into specific components of the user interface, e.g., graphs, parameter boxes, buttons or icons. The document US 2015/106121 A1 describes an alarm system with an escalation process as a function of the responses of the alarm generator to notifications (alarm information) by the alarm-communicating system. The alarm information used contains here information on (information relating to) a physiological parameter. Physiological information is sent with the alarm and alarm information is first sent by the alarm-communicating system (e.g., alarm server).

The document US 2009/326340 A1 shows an alarm system with a physiological monitor as an alarm source, in which system the communicating system prompts the signal generator of the alarm source to acoustically reproduce a previously silent alarm. Alarm generation at a monitor may depend on the alarm-communicating system in case of an error.

### SUMMARY OF THE INVENTION

Therefore, there is a need for creating an improved communication concept for an alarm system. This need is met by an alarm source, an alarm server, an alarm generator and an alarm system, processes and computer programs according to the invention.

Exemplary embodiments are based on the central idea of making possible a reliable distribution of clinical and other alarms to recipients and/or groups of recipients, without the actual availability of the recipient/recipients being known in advance. The individual alarm may be both a primary alarm, i.e., the alarm source itself does not output an acoustic alarm, or a secondary alarm, i.e., both the alarm source and a remote alarm generator signal the alarm. Alarms are correspondingly forwarded to recipients, without these having to be known in advance. Exemplary embodiments can make the sending of alarm messages more robust with respect to acutely developing disturbances due to the fact that the alarm server does not have to know the status of all recipients at a central location in advance (single point of failure), but recipient terminals themselves are able to get ad hoc alarm messages for themselves and subsequently to send a signal. The transmission and escalation process of the alarm messages can make use of low transportation latencies.

A prior availability check by the server may be eliminated according to the invention. Since no availability information is needed in advance for transmitting alarm messages to the terminal at least in some exemplary embodiments, it is also unnecessary to store corresponding data on the server. This reduces the risk of using obsolete or incorrect status information relating to the state of availability of the terminals for forwarding alarms. Should these data nevertheless be stored, this can happen for meeting logging obligations. This is not a prerequisite for effectively forwarding an alarm. Moreover, the system becomes more secure against server failures in some exemplary embodiments, because the recipient terminals can receive a configurable list of failed servers, which list can be maintained remotely, so that the receiving terminals are capable of changing automatically over to a back-up server in case of an acute network partitioning.

Exemplary embodiments create a device for an alarm server. The device comprises one or more interfaces for communication with one or more alarm sources and with one or more alarm generators, and further a memory for storing information on one or more alarms. The device comprises, moreover, a control module for controlling the one or more interfaces and the memory. The control module is configured to receive an indication relating to an alarm via the one or more interfaces from an alarm source and to store information on the alarm (information relating to the alarm) in the memory. The control module is further configured to receive an alarm request from an alarm generator via the one or more interfaces and to provide information for the alarm generator after receiving the alarm request if information on the alarm is stored in the memory.

Exemplary embodiments also create a device for an alarm source. The device has one or more interfaces for communication with an alarm server, and a monitoring device for monitoring a patient parameter and/or a status of a medical device and for providing an alarm indication when the patient parameter meets a predefined condition. The device further comprises a control module for controlling the one or more interfaces and the monitoring device. The control module is configured to forward an alarm indication provided by the monitoring device to the alarm server via the one or more interfaces. The control module is configured to trigger a local alarm at the alarm source after receiving an alarm indication from the monitoring device if no confirmation of the receipt of the alarm indication by an alarm generator was received after a predefined time, T2.

Exemplary embodiments also create a device for an alarm generator. The device has one or more interfaces for communication with an alarm server and an output device for outputting information on an alarm to a user. The device comprises a control module for controlling the one or more interfaces and the output device. The control module is further configured to poll an alarm server for the presence of an alarm via the one or more interfaces and to receive information on the presence of an alarm from the alarm server being polled. The control module is further configured to signal the presence of the alarm to the user via the output device upon receipt of the information on the presence of an alarm from the alarm server.

Exemplary embodiments also create an alarm system with an exemplary embodiment of a device for an alarm server, with an exemplary embodiment of a device for an alarm source and with an exemplary embodiment of a device for an alarm generator.

Exemplary embodiments can thus also make an efficient communication of alarms or alarm information possible without prior availability check or heartbeat signals. For example, alarm information from the device for the alarm server can be stored in an associative memory as a memory device.

In some exemplary embodiments, the indication of the alarm source may indicate a state of alarm of the alarm source, and the control module of the device for the alarm server may be configured to store status information on a state of alarm of the alarm source in the memory. Such status information may then, for example, be polled and searched or updated with little effort. The control module of the device for the alarm server may be configured to provide or to confirm information on the alarm to the alarm generator in the pull method for the alarm generator. The control module of the device for the alarm generator may be configured analogously to poll the alarm server for the presence of an alarm in a pull method for the alarm generator. A greater effort for signaling for the alarm generator that would exceed this can thus be eliminated in some exemplary embodiments.

The control module of the device for the alarm server may be configured to poll the alarm source for the indication on the alarm according to a pull method for the alarm source and/or to receive the indication on the alarm according to a push method for the alarm source. The control module of the device for the alarm source may be configured to provide the alarm indication on the alarm for the alarm server upon polling by the alarm server according to a pull method for the alarm source and/or to provide the alarm indication on the alarm for the alarm server according to a push method for the alarm source. Consequently, exemplary embodiments offer a plurality of signaling possibilities between the alarm source and the alarm server.

In some additional exemplary embodiments, the control module of the device for the alarm server is configured to associatively store in the memory with the indication one or more components of the group comprising an identification of the alarm source, properties of the alarm, a category of the alarm, a location of the alarm, or a priority of the alarm and to forward it to the alarm generator with confirmation of the alarm. Exemplary embodiments can thus make possible an efficient management and alarm generation due to additional information. Moreover, the control module of the device for the alarm server may be configured to trigger forwarding to additional alarm generators and/or to trigger a local alarm at the alarm server after confirmation of the presence of an alarm to the alarm generator if no confirmation is received from the alarm generator after a predefined time, T5, that the presence of an alarm has been acknowledged by a user. Exemplary embodiments can thus provide a plurality of alarm levels.

In some exemplary embodiments, the monitoring device of the device for the alarm source may be configured to monitor the patient parameter taking into account at least one sensor signal of a medical device and/or at least one operating parameter of an actuator of a medical device concerning the predefined condition. Exemplary embodiments may thus create an automated alarm generation concept for a patient. In further exemplary embodiments, the predefined time, T2, may correspond to a predefined second time. The control module of the device for the alarm source may be configured to trigger a local alarm at the alarm source upon receipt of an alarm indication from the monitoring device if no confirmation was received from the alarm server after a predefined first time, T1, that the alarm indication was received by the alarm server. The control module of the device for the alarm source may be configured to trigger a local alarm at the alarm source upon receipt of an alarm indication from the monitoring device if no confirmation was obtained from the alarm server after a predefined third time, T3, that the alarm indication had been acknowledged by a user. The control module of the device for the alarm source may be configured to trigger a local alarm at the alarm source upon receipt of an alarm indication from the monitoring device if no acknowledgment of the alarm indication by a user was received at the alarm source after a predefined fourth time, T4, and the patient parameter still meets the predefined condition. Exemplary embodiments can thus monitor the communication process over the predefined times and ensure corresponding alarm generations if disturbances or errors are present in the communication process.

In some additional exemplary embodiments, the control module of the device for the alarm source may be configured to adapt at least one of the predefined times, T1, T2, T3, T4, according to a priority of the alarm. Exemplary embodiments may thus allow an adapted alarm generation.

The control module of the device for the alarm generator may be configured to poll for the presence of alarm at an alarm server at regular configurable time intervals in an event-based manner or as a function of an operating state of the alarm generator. Exemplary embodiments can thus make possible a process-adapted and/or time-adapted alarm generation. The control module of the device for the alarm generator may be configured in additional exemplary embodiments to trigger a local alarm at the alarm generator upon receipt of the information or a confirmation of the presence of an alarm from the alarm server if the presence of an alarm was not acknowledged by the user after a predefined time. Exemplary embodiments can thus also make an alarm generation by the alarm generator possible in case of a disturbance in communication. Analogously to the above description, the predefined time may be adapted according to the priority of the alarm.

Exemplary embodiments also create a process for an alarm server. The process comprises the receipt of an indication on an alarm from an alarm source and the storage of information on the alarm. The process further comprises the receipt of an alarm request from an alarm generator and the supply of information on the alarm to the alarm generator if information on the alarm is stored.

Exemplary embodiments also create a process for an alarm source. The process comprises the monitoring of a patient parameter and/or of a status of a medical device and the provision of an alarm indication if the patient parameter meets a predefined condition. The process also comprises the forwarding of a provided alarm indication to an alarm server. The process also comprises the triggering of a local alarm if the alarm server did not receive a confirmation of the receipt of the alarm indication by an alarm generator after a predefined time, T2.

Exemplary embodiments also create a process for an alarm generator. The process comprises polling for the presence of an alarm at an alarm server and the receipt of information on the presence of an alarm upon polling by the alarm server. The process further comprises signaling of the presence of an alarm to a user.

Another exemplary embodiment is a computer program with a program code for executing at least one of the processes being described here, if the program code is carried out on a computer, a processor or a programmable hardware component.

Further advantageous embodiments will be described in more detail below on the basis of the exemplary embodiments shown in the drawings, even though the embodiments are not generally limited, as a whole, to these exemplary embodiments. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a schematic view showing an exemplary embodiment of a device for an alarm server, an exemplary embodiment of a device for an alarm source, an exemplary embodiment of a device for an alarm generator, and an exemplary embodiment of an alarm system;

FIG. 2 is a schematic view showing another exemplary embodiment of an alarm system with two alarm sources, with an alarm server and with three alarm generators;

FIG. 3 is a schematic view showing an exemplary embodiment of an alarm system with an illustration of exchanged messages;

FIG. 4 is a schematic view showing an exemplary embodiment of an alarm system with a failure of an alarm server;

FIG. 5 is a schematic view showing an exemplary embodiment of an alarm system with a failure of an alarm generator;

FIG. 6 is a schematic view showing an exemplary embodiment of an alarm system with the absence of an acknowledgment by a user at the alarm generator;

FIG. 7 is a schematic view showing an exemplary embodiment of an alarm system with the absence of an acknowledgment by a user at the alarm source;

FIG. 8 is a block diagram of a flow chart of a process for an alarm source in an exemplary embodiment;

FIG. 9 is a block diagram of a flow chart of a process for an alarm server in an exemplary embodiment;

FIG. 10 is a block diagram of a flow chart of a process for an alarm server and for an alarm generator in an exemplary embodiment;

FIG. 11 is a block diagram of a flow chart of a process for an alarm generator in an exemplary embodiment;

FIG. 12 shows a block diagram of a flow chart of a process for an alarm server in another exemplary embodiment;

FIG. 13 is a block diagram of a flow chart of a process for an alarm source in another exemplary embodiment; and

FIG. 14 is a block diagram of a flow chart of a process for an alarm generator in another exemplary embodiment.

### DESCRIPTION OF PREFERRED EMBODIMENTS

Referring to the drawings, different exemplary embodiments are described in more detail with reference to the attached drawings, in which some exemplary embodiments are shown.

Identical reference numbers may designate identical or comparable components in the following description of the attached figures, which show only some exemplary embodiments. Further, summary reference numbers may be used for components and objects that appear multiple times in an exemplary embodiment or in a drawing, but are described jointly concerning one or more features. Components or objects that are described with the same or summary reference numbers may have the same configuration but optionally also different configurations in respect to individual features, a plurality of features or all features, for example, their dimensioning, unless something different explicitly or implicitly appears from the description. Optional components are represented by broken lines or arrows in the figures.

Even though exemplary embodiments may be modified and varied in different ways, exemplary embodiments are shown in the figures as examples and are described herein in detail. It shall, however, be made clear that exemplary embodiments are not intended to be limited to the particular disclosed forms, but exemplary embodiments shall rather cover all functional and/or structural modifications, equivalents and alternatives, which are within the scope of the present invention. Identical reference numbers designate identical or similar components in the descriptions of all figures.

Please note that a component, which is described as being “connected” or “coupled” to another component, may be connected or coupled to the other component directly or components located between them may be present. If, by contrast, a component is described as being “directly connected” or “directly coupled” to another component, no components that would be located between them are present. Other terms, which are used to describe the relationship between components, should be interpreted in a similar manner (e.g., “between” vs. “directly between,” “adjoining” vs. “directly adjoining,” etc.).

The terminology that is being used is intended only to describe certain exemplary embodiments and shall not limit the exemplary embodiments. As is being used here, the singular forms “a, one” and “the” shall also include the plural forms unless the context unambiguously indicates something else. Further, it shall be made clear that such terms as, e.g., “contains,” “containing,” “has,” “comprises,” “comprising” and or “having,” as herein used, indicate the presence of said features, integers, steps, work processes, elements and/or components, but they do not exclude the presence or the addition of one or more features, integers, steps, work processes, elements, components and/or groups thereof.

Unless defined otherwise, all the terms being used here (including technical and scientific terms) have the same meaning that one of average skill in the art in the field to which the exemplary embodiments belong attributes to them. Further, it shall be made clear that terms, e.g., those that are defined in generally used dictionaries, are to be interpreted as if they have the meaning that is consistent with their meaning in the context of the relevant technology and are not to be interpreted in an idealized or excessively formal sense, unless this is expressly defined here.

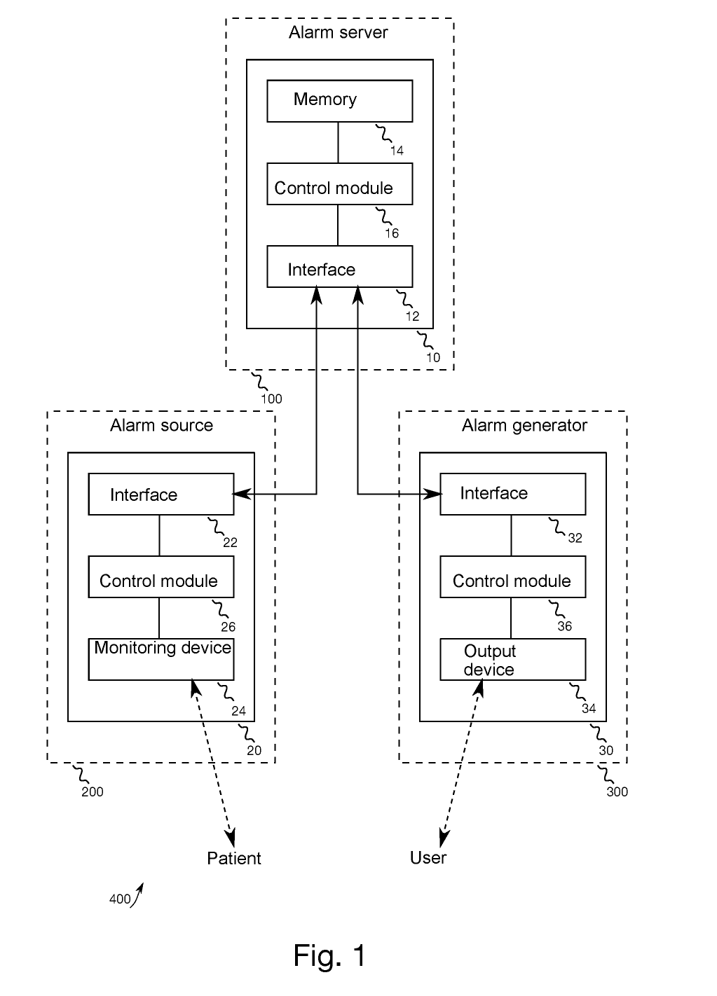

FIG. 1 shows an exemplary embodiment of a device 10 for an alarm server 100. Exemplary embodiments also create an alarm server 100 (shown by broken lines as an optional component in FIG. 1) with a device 10. The device 10 comprises one or more interfaces 12 for communication with one or more alarm sources 200 and with one or more alarm generators 300. The device 10 comprises, moreover, a memory 14 for storing information on one or more alarms. The device further comprises a control module 16 for controlling the one or more interfaces 12 and the memory 14. The control module 16 is further configured to receive an indication on an alarm via the one or more interfaces 12 from an alarm source 200 and to store information on the alarm (information relating to the alarm) in the memory 14. The control module 16 is configured to receive an alarm request from an alarm generator 300 via the one or more interfaces 12 and to provide information on the alarm for the alarm generator 300 upon receipt of the alarm request if information on the alarm is stored in the memory 14.

Further, FIG. 1 shows an exemplary embodiment of a device 20 for an alarm source 200. Exemplary embodiments also create an alarm source 200 (shown by broken lines as an optional component in FIG. 1) with a device 20. The device 20 comprises one or more interfaces 22 for communication with an alarm server 100 and one or more monitoring devices 24 for monitoring a patient parameter and/or a status of a medical device and for providing an alarm indication if the patient parameter meets a predefined condition. The device comprises, moreover, a control module 26 for controlling the one or more interfaces 22 and the monitoring device 24. The control module 26 is configured to forward an alarm indication provided by the monitoring device 24 to the alarm server 100 via the one or more interfaces 22. The control module 26 is configured to trigger a local alarm at the alarm source 200 upon receipt of an alarm indication from the monitoring device 24 if confirmation by the alarm server 100 of the receipt of the alarm indication by an alarm generator 300 was not received after a predefined time, T2.

FIG. 1 shows, moreover, an exemplary embodiment of a device 30 for an alarm generator 300. Exemplary embodiments also create an alarm generator 300 (shown by broken lines as an optional component in FIG. 1) with a device 30. The device 30 comprises one or more interfaces 32 for communication with an alarm server 100 and an output device 34 for outputting information on an alarm to a user. The device 30 comprises, moreover, a control module 36 for controlling the one or more interfaces 32 and the output device 34. The control module 36 is further configured to poll an alarm server 100 for the presence of an alarm via the one or more interfaces 32 and to receive information on the presence of an alarm from the alarm server 100 upon request. The control module 36 is further configured to signal the presence of the alarm via the output unit 34 to the user upon receipt of the information on the presence of an alarm.

FIG. 1 shows, moreover, an exemplary embodiment of an alarm system 400 with a device 10 for an alarm server 100, with a device 20 for an alarm source 200 and with a device 30 for an alarm generator 300.

In exemplary embodiments, said interfaces 12, 22, 32 are used for the communication of the components with one another, for example, by means of digital or analog signals. The one or more interfaces 12, 22, 32 may be, for example, network interfaces and correspond to one or more inputs and/or one or more outputs for receiving and/or transmitting information, e.g., in digital bit values, analog signals, magnetic fields, based on a code, within a module, between modules, or between modules of different entities. This may happen in exemplary embodiments in a wired or wireless manner, depending on how the connection of the components to one another is configured. Some variants of network interfaces, which may be provided in exemplary embodiments, are RJ45, Ethernet, Wireless Local Area Network (WLAN), 802.11, Bluetooth (BT), mobile telephone, optical communication (infrared), acoustic communication (ultrasound), etc.

The memory 14 may correspond in exemplary embodiments to any desired memory or memory component, and volatile and nonvolatile memories are conceivable. Examples are hard drive spaces, flash memories, solid-state memories, random access memory (RAM) memories, etc. Exemplary embodiments are not limited to a certain memory technology, and any memory that is suitable for the storage of information may be used.

A control module 16, 16, 36 may correspond in exemplary embodiments to any desired controller or processor or a programmable hardware component. For example, a control module 16, 26, 36 may also be embodied as software, which is programmed for a corresponding hardware component. A control module 16, 26, 36 may thus be implemented as programmable hardware with correspondingly adapted software. Any desired processors, such as digital signal processors (DSPs) or processors in general may be used. Exemplary embodiments are not limited to a certain type of processor. Any desired processor or even a plurality of processors may be provided for implementing a control module 16, 26, 36.

The monitoring device 24 may be, for example, a patient monitor, which monitors vital parameters; a sensor of an electrocardiogram (EKG), a sensor of a ventilator, etc. The monitoring device 24 may also be configured, as an alternative or in addition, for monitoring a status of a medical device, for example, for monitoring a tube or a tube connection, a power supply, an overheating, a readiness of the medical device to operate in general, etc. Thus, any sensor or any detection device is suitable for monitoring a medical device or for detecting a patient parameter, i.e., a value relevant for an alarm generation or a variable relevant for an alarm generation.

The output device 34 may be, for example, a display, a monitor, an indicator light, a loudspeaker, a haptic device, etc., which is suitable for alerting a user to an existing alarm or to signal the alarm to that user.

An indication of an alarm may be in some exemplary embodiments a single binary value, i.e., a bit, which indicates that an alarm is present. In other exemplary embodiments, the indication may also comprise additional information, as will be explained in more detail below. The indication may be represented, for example, by a signal flank or a predefined signal value. The alarm request may also comprise, depending on the communication protocol, one or more values and may also comprise additional information, for example, an identification, an address, a code, etc., in addition to a simple signaling that a request is present.

A local alarm of a respective component is consequently defined here as an action to alert persons located in the area surrounding the respective component, e.g., clinical staff, to the component or to the alarm, for example, optical and/or acoustic signals may be provided in this connection. A local alarm is not limited here to the respective component, so that additional alarm generation devices located in the surrounding area can also be alarmed in case of a local alarm in a wired or also wireless manner.

Exemplary embodiments make possible an efficient communication effort, because the alarm generator 300 proactively polls the alarm server 100 for existing alarms. An initialization phase of the alarm generator 300 until readiness to operate can be kept short as a result.

Exemplary embodiments may also do here without a dedicated heartbeat signal (also called keep-alive signal, e.g., context-maintaining recurring signal), but an alarm-generating terminal (alarm generator 300) may poll the alarm-communicating and -managing system (alarm server 100) for certain alarms at regular or irregular time intervals. The alarm-communicating system (alarm server 100) thus uses no heartbeat signal to detect the status of an alarm generator. A failure of an alarm generator 300 can be detected in some exemplary embodiments on the basis of a correspondingly run-down) timer (time measuring device, time value measuring device).

Some exemplary embodiments do not require a transmission of vital data, nor is the transmission initiated by the alarm-communicating system (push method), but the information is requested actively (pull method). The control module 16 of the device 10 for the alarm server 100 may be configured to provide or confirm the information on the alarm to the alarm generator 300 in a pull method for the alarm generator 300. Exemplary embodiments can thus provide an increased reliability or failure safety of the system, because they are based, due to the timer used, on the intrinsic safety of all individual components and all components are able to send technical alarm signals independently in case of a (partial) failure. Therefore, no prior availability check of the recipients is necessary for the alarm distribution in exemplary embodiments. The selection of the recipient in escalation scenarios can likewise take place without a prior availability check by the alarm server 100. An active monitoring of the availability by dedicated heartbeat signals of the recipients may also be eliminated.

In some additional exemplary embodiments, a recipient cannot display the raw version of the data, but the alarm server 100 analyzes these and gives an “adapted alarm signal” or stores a data component, which indicates an adapted state of alarm. In at least some exemplary embodiments, the indication of the alarm source 200 can generally display a state of alarm of the alarm source 200. The control module 16 on the side of the alarm server 100 may be configured to store status information on a state of alarm of the alarm source in the memory 14. The control module 16 is configured to associatively store with the indication one or more components of the group comprising an identification of the alarm source 200, properties of the alarm, a category of the alarm, a location of the alarm, or a priority of the alarm in the memory 14 and to forward it to the alarm generator 300 with confirmation of the alarm.

The data component may be written in a so-called associative memory, in which case this associative memory also stores properties of the data components, so that a search can be performed for them. This may also be carried out, for example, with a fuzzy search, during which degrees of similarity are determined. For example, the alarm server may keep ready an adapted alarm until this is “collected” by the alarm generator 300 in the pull method. Exemplary embodiments do not necessarily transmit alarm and vital data. A transmission is initiated by the alarm generator 300. Exemplary embodiments can create an intrinsically safe system 400, for example, by the intrinsic, independent safety functions of the alarm source.

Some exemplary embodiments will be explained below. The alarm source (AQ) 200 analyzes alarm conditions, taking into account at least one sensor signal of a medical device and/or at least one operating parameter of an actuator of a medical device, e.g., the vital parameters (physiological parameters) of one or more patients and/or of a status of a medical device, e.g. a gas concentration measured value within a ventilator or anesthesia device or, e.g., a drive frequency of a breathing gas delivery unit of a ventilator, and can signal detected alarm conditions. An alarm condition is defined as the state of an alarm system when a hazardous situation is detected (the patient parameter meets a predefined condition). In some other exemplary embodiments, the monitoring device 24 is configured to monitor the patient parameter while taking into account at least one sensor signal of a medical device and/or at least one operating parameter of an actuator of a medical device with respect to the predefined condition.

The alarm server (AS) 100 manages and stores the alarm messages sent by the alarm sources. It makes possible the connection of alarm information and the generation of new alarms. The alarm server 100 can operate in the push mode and the pull mode with respect to the communication between the alarm source 200 and the alarm server 100. Alarms of the AQs 200 are sent to the alarm server 100 in the push mode. The alarm server 100 actively collects the alarms from the AQs 200 in the pull operation.

Consequently, the control module 16 of the device 10 for the alarm server 100 may be configured in some exemplary embodiments to poll the indication on the alarm at the alarm source 200 according to a pull method for the alarm source 200 and/or to obtain the indication on the alarm according to a push method for the alarm source 200. Analogously, the control module 26 for the device 20 of the alarm source 200 may be configured to provide the alarm indication on the alarm for the alarm server 100 upon request by the alarm server 100 according to a pull method for the alarm source 200 and/or to provide the alarm indication on the alarm for the alarm server 100 according to a push method for the alarm source 200.

The alarm generator (AG) 300 actively receives alarms from the alarm server 100 according to a pull method and signals them at the user. The alarm generator 300 may have either a stationary or a mobile configuration, e.g., it may be a tablet, mobile telephone, pager, etc. The control module 36 of the device 30 for the alarm generator 300 may be configured to poll the alarm server 100 for the presence of an alarm according to a pull method for the alarm generator 300.

A user (B) will hereinafter be defined especially as clinical staff, a nurse, a physician or a technician, who is informed by the alarm generator 300 about an alarm condition in order to perform an action at the alarm source 200 (e.g., at the patient monitor). User groups (BG) may comprise a plurality of users, who possess at least one property in common, e.g., assigned patient, location, qualification or area of responsibility. Users may be in a plurality of user groups at the same time, and user groups may overlap.

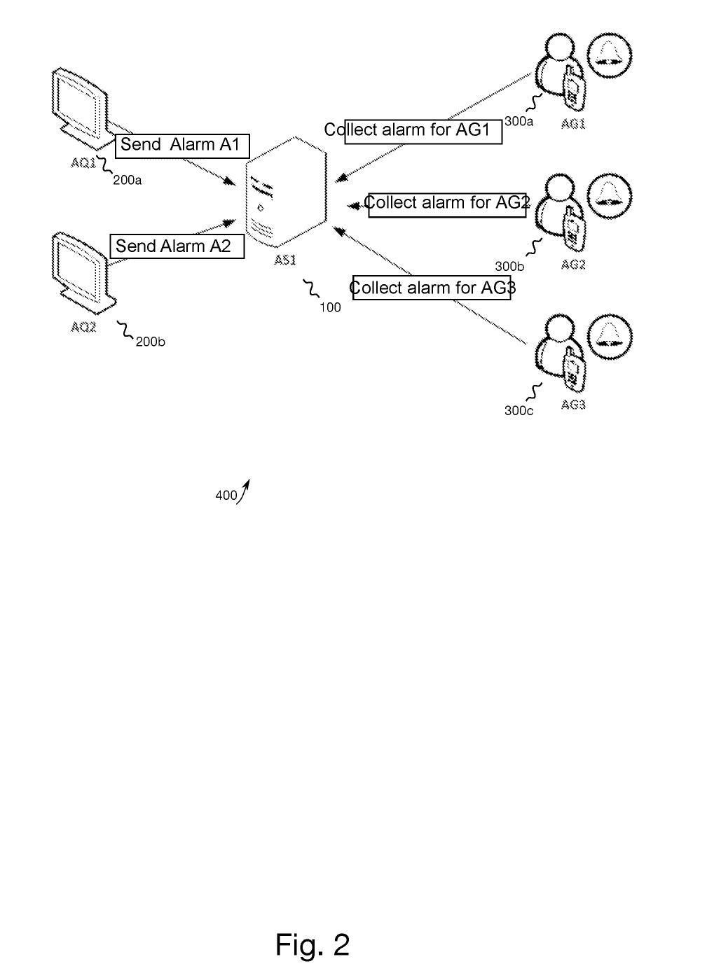

FIG. 2 shows another exemplary embodiment of an alarm system 400 with two alarm sources 200a, 200b (AQ1, AQ2), with an alarm server 100 (AS1) and with three alarm generators 300a, 300b, 300c (AG1, AG2, AG3). In general, at least one alarm source 200, at least one alarm server 100 and at least one alarm generator 300 are present in the alarm system 400. Each alarm source 200a, 200b monitors the vital parameters and/or the correctness of the function of the alarm source 200a, 200b. In case of an alarm condition at one of the alarm sources 200a, 200b, an alarm message is sent to the alarm server 100, which stores this together with the identification of the alarm source 200a, 200b. The stored alarm messages are collected actively from the alarm generators 300a, 300b, 300c, and a corresponding alarm signal is triggered on the device. The alarm messages may be transported and/or (temporarily) stored in both the coded and uncoded form. The alarm generators 300a, 300b, 300c may be implemented in exemplary embodiments as mobile terminals assigned to clinical staff members, as this is indicated in the figures.

The alarm sources 200a, 200b monitor the vital parameters of a patient and/or the correctness of the function of the device as well as of the connected auxiliary means (e.g., auxiliary devices, sensors, tubes, etc.). All the devices that have a monitoring and/or therapeutic function at the patient, e.g., patient monitors, EKGs, ventilators, anesthesia devices, syringe pumps and others, belong to the alarm sources 200. It may be useful in case of an alarm condition not to trigger any acoustic alarm at the device at first, but to forward an alarm message to one or more alarm generators 300, e.g., via an alarm server 100, according to the aforementioned pull method, and these alarm generators 300 will then trigger an alarm signal at a user. The alarm source 200 has at least one timer, via which the communication to and from the other components is secured. If the alarm source 200 cannot forward an alarm message to an alarm generator 300 based on a failure of the device (e.g., of the alarm server 100), the alarm is triggered at the alarm source 200 after the timer has run down (predefined time T2).

All the alarm messages sent by the alarm sources 200 are stored in a memory 14 of the alarm server 100, preferably called associative memory, together with the corresponding alarm properties. The alarm properties may either be transmitted together from the alarm source 200 or they are stored on the alarm server 100. A concrete device alarm (e.g., excessively low heartbeat) is represented by an unambiguous alarm identification. The alarm properties contain all the data that are associated with the alarm, for example, the priority of the alarm, the location of the patient, or also the classification as clinical or technical alarms. The alarm properties are used as codes for searching for a suitable alarm message in the memory 14 when an alarm massage shall be received from an alarm generator 300. As soon as an alarm message is assigned to an alarm generator 300, it is marked with the identification of the alarm source 200 and of the alarm generator 300. It can be ensured thereby that an alarm is assigned at a time to only one alarm generator 300 (if desired), unless there are other reasons against doing so.

The alarm messages of the alarm sources 200 are preferably analyzed by the alarm server 100, so that the alarms received directly from the alarm sources 200 are not stored in the memory 14 of the alarm server 100, but the alarm server 100 analyzes a plurality of alarm messages in order to prepare from this an “adapted” alarm message and to then store the adapted alarm message in the so-called associative memory and to keep the adapted alarm message available for the pull method.

In other words, for example, an associative memory, in which the alarm message can be found on the basis of alarm properties, is used in order to carry out the search for a fitting alarm message efficiently. The search is not limited now to a complete agreement of the properties; alarm messages, in which only individual properties agree or these are in a certain range (e.g., when using localization properties), shall rather be found based on a so-called “fuzzy search.”

An alarm generator 300 assumes, as a primary alarm device, the function of user notification, so that no acoustic alarm generation is at first necessary at the alarm source 200 in normal operation. As an alternative, the alarm generator 300 may be used as a secondary alarm device, in which case the alarms can be triggered simultaneously at different components (e.g., also at the alarm source 200 or at another alarm generator 300). The use may be configured as a primary or secondary alarm device in exemplary embodiments. The alarm generator functionality may be implemented on different types of devices, for example, tablet computers, pagers or even mobile telephones. Implementation on any terminal that offers a certain basic functionality (triggering alarm, confirming alarm, etc.) is conceivable, in principle.

During the operation, the alarm generator 300 requests alarm messages from the alarm server 100, for example, at regular, preferably configurable intervals, or at irregular intervals (keyword: random access method over the network), or depending on an operating state of the alarm generator 300 (for example, the physician is currently busy and sets the device on “standby”), and user characteristics may additionally be transmitted to the alarm server 100. The control module 36 of the device 30 for the alarm generator 300 may be configured to poll the alarm server 100 for the presence of alarms at regular, configurable time intervals, in an event-based manner or as a function of an operating state of the alarm generator 300.

User characteristics may be, e.g., the distance of the user from the patient or even his core duties. These are used to adapt the selection of an alarm message to the corresponding user in an optimal or at least improved manner.

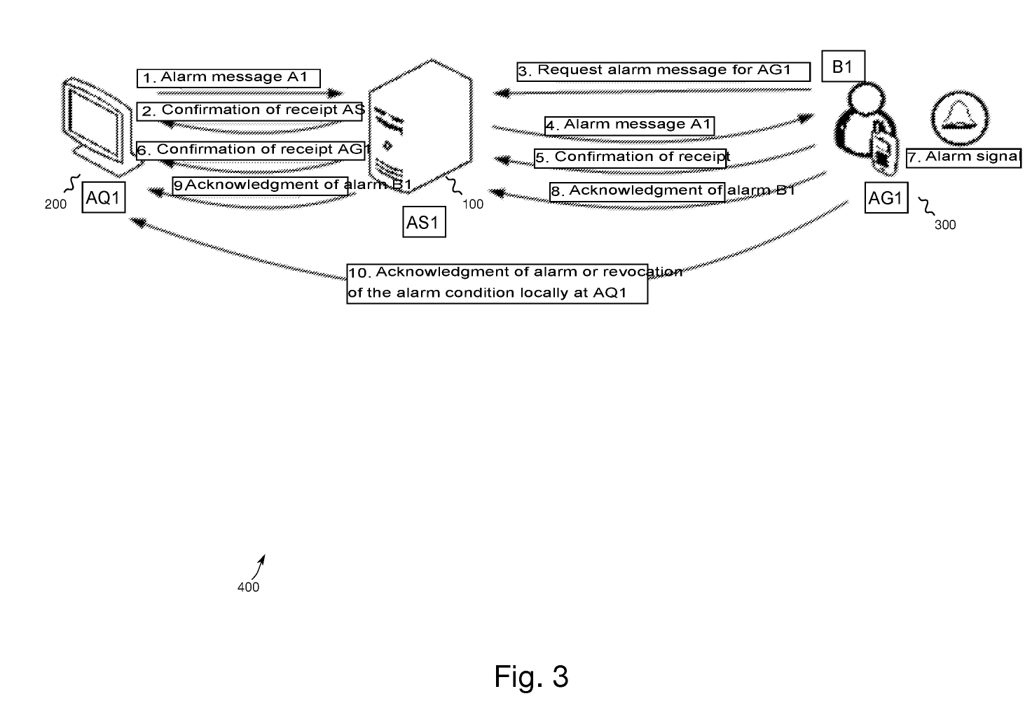

FIG. 3 shows another exemplary embodiment of an alarm system 400 with an illustration of exchanged messages. The description of the complete alarm system as well as the communication between the individual components, already described above, will be presented below in detail on the basis of individual scenarios.

Review of the timers used:

T1 (Alarm Source 200):
The alarm server 100 has received the alarm report. The control module 26 of the device 20 of the alarm source 200 may be configured to trigger a local alarm at the alarm source 200 upon receipt of an alarm indication from the monitoring device 24 if no confirmation is received after a predefined first time, T1, that the alarm indication has been received by the alarm server 100.

T2 (Alarm Source 200):
The alarm generator 300 has received the alarm report. This timer was already described above as the predefined time of the alarm source 200. If the alarm source 200 cannot forward an alarm message to an alarm generator 300 based on a failure of a device (e.g., of the alarm server 100), the alarm is triggered at the alarm source 200 after the rundown of the timer, T2. This predefined time, T2, may correspond to a second predefined time (T1 now corresponds to the first predefined time).

T3 (Alarm Source 200):
The user has acknowledged the alarm signal at the alarm generator 300. The control module 26 of the device 20 of the alarm source 200 may be configured to trigger a local alarm at the alarm source 200 upon receipt of an alarm indication from the monitoring device 24 if no confirmation was received from the alarm server 100 after a predefined time, T3, that the alarm indication has been received by a user.

T4 (Alarm Source 200):
The user has acknowledged the alarm signal at the alarm source. The control module 26 of the device 20 of the alarm source 200 may be configured to trigger a local alarm at the alarm source 200 upon receipt of an alarm indication from the monitoring device 24 if no acknowledgment of the alarm indication by a user was received at the alarm source 200 after a predefined time, T4, and the patient parameter still continues to meet the predefined condition.

T5 (Alarm Server 100):
The user has acknowledged the alarm signal at the alarm generator. The control module 16 of the device 10 of the alarm server 100 is configured to trigger forwarding to additional alarm generators 300 and/or a local alarm at the alarm server 100 after confirmation of the presence of an alarm at the alarm generator 300 if the alarm generator 300 does not confirm the acknowledgment of the presence of an alarm by a user after a predefined time, T5. The control module 36 of the alarm generator 300 may also be configured in some additional exemplary embodiments to trigger a local alarm at the alarm generator 300 upon receipt of the information or after a confirmation of the presence of an alarm from the alarm server 100 if the presence of an alarm is not acknowledged by the user after a predefined time. The control module 36 may further be configured to adapt the predefined time according to the priority of the alarm.

In the exemplary embodiments explained on the basis of FIG. 3, the alarm source AQ1 200 detects the alarm condition A1, the alarm generator 300 AG1 generates an alarm and the user B1 receives the alarm. This scenario describes the normal operation of the alarm system 400 in an exemplary embodiment. AQ1 200 detects an alarm condition and sends an alarm message to AS1 100 (1). This confirms the message at AQ1 200 (2). Independently from this, AG1 300 polls AS 100 for possible alarm messages (3). AS1 100 sends the corresponding alarm message A1 over to AG1 300 (4), which confirms the message at AS1 100 (5). AS1 100 forwards the confirmation of receipt to AG1 200 (6). At the same time, the alarm signal is triggered by AG1 300 at the user B1 (7); as soon as this confirms the alarm signal, the acknowledgment of the alarm is forwarded to AS1 100 (8) and from here to AQ1 200 (9). The user B1 acknowledges the alarm or eliminates the cause of the alarm on the spot (10).

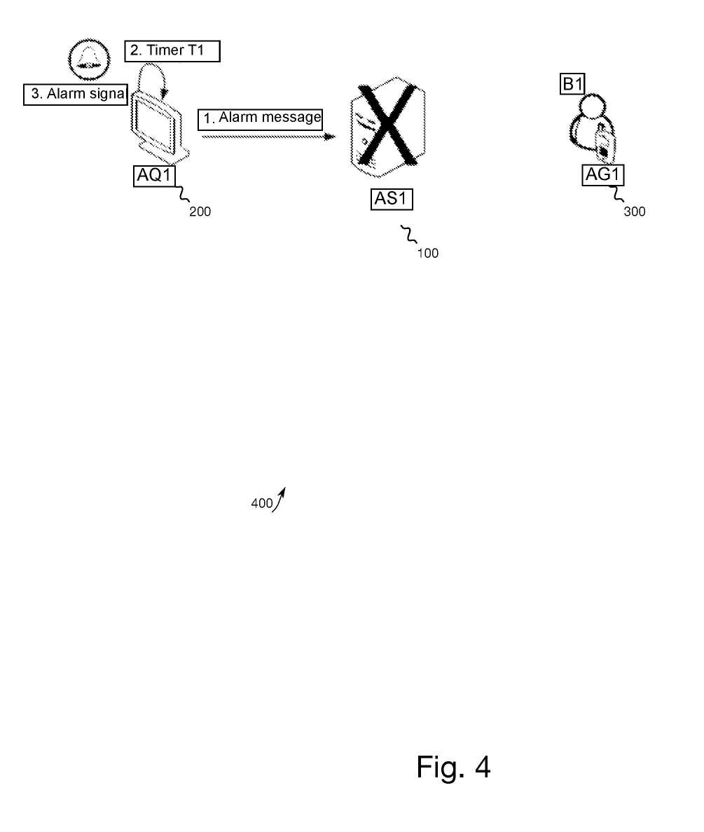

FIG. 4 shows another exemplary embodiment of an alarm system 400 with a failure of an alarm server 100. The alarm server 100 does not receive any alarms from AQs 200 in this exemplary embodiment. To ensure the triggering of an alarm signal in time in case of a failure of the alarm server 100, the acceptance of the alarm message by the alarm server 100 is monitored based on a timeout. If, for example, AQ1 200 communicates the alarm message A1 to AS1 100 (1), the timer T1 is started at the same time (2). If no confirmation of receipt was received by the alarm server 100 before the rundown of the timer, a local alarm is triggered at the alarm source 200 (3).

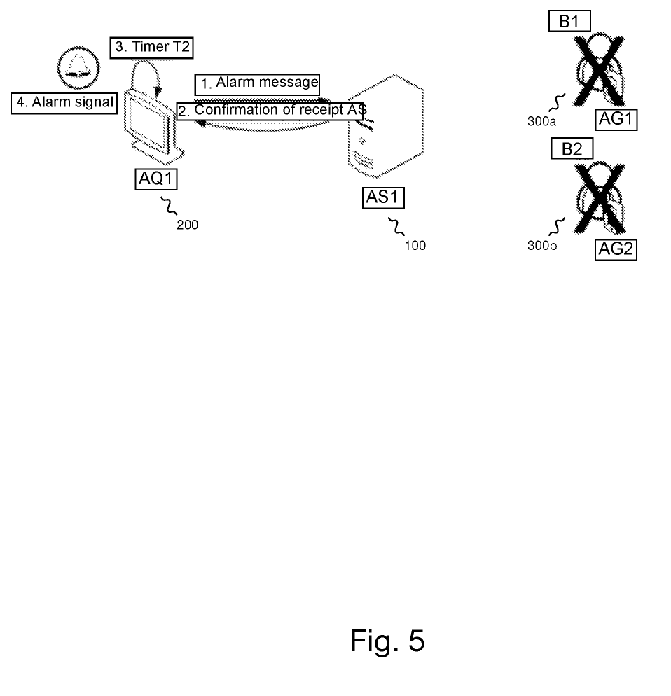

FIG. 5 shows an exemplary embodiment of an alarm system 400 with a failure of an alarm generator 300. AQ1 200 detects in this exemplary embodiment the alarm condition and sends an alarm message to the alarm server 100. However, the alarm is not received by any alarm generator 300. AQ1 200 detects the alarm condition and sends the alarm message to AS1 100 (1), which confirms this (2). AQ1 200 starts the timer T2, by means of which the receipt of the alarm by an alarm generator 300a, 300b is monitored. If T2 expires and no positive feedback of an alarm generator 300a, 300b was received by AS1 100 nor forwarded to AQ1 200, the alarm signal is triggered at the alarm source 200 (4).

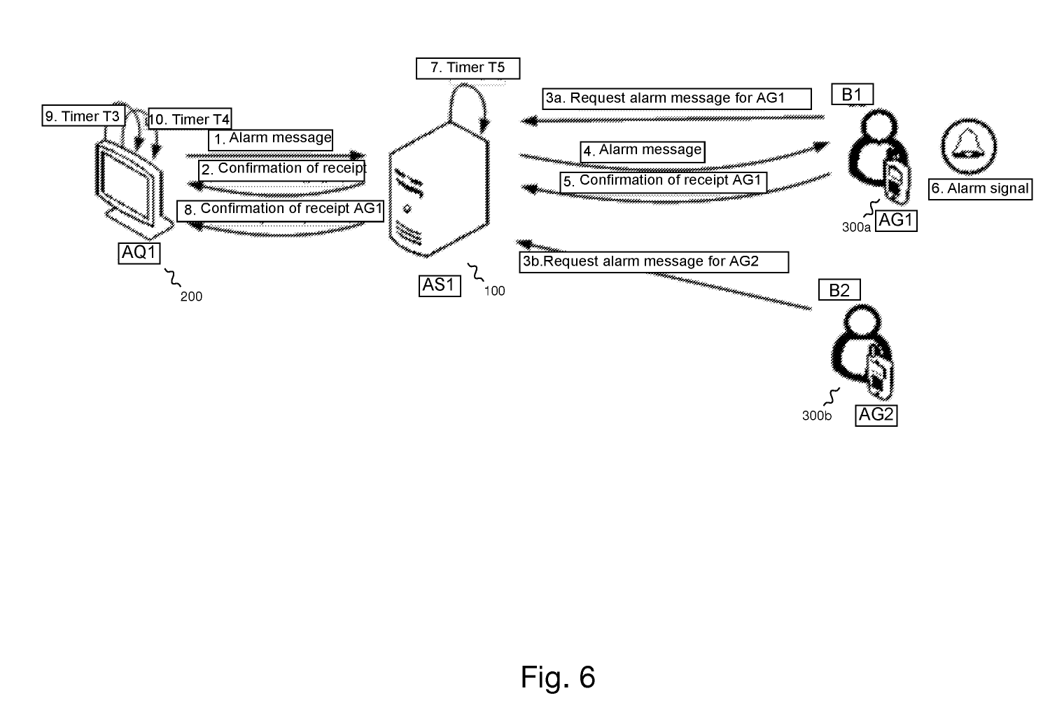

FIG. 6 shows an exemplary embodiment of an alarm system 400 with the absence of acknowledgment by a user at the alarm generator 300. AQ1 200 detects in this exemplary embodiment the alarm condition, a user B1 is alarmed via AG1 300a and the user B1 receives the alarm, but the user B1 does not acknowledge the alarm at the alarm generator AG1 300a. Analogously to the previous scenario, AQ1 200 detects the alarm condition and sends an alarm message to AS1 100 (1), which confirms (2) same. Independently from this, AG1 300a and AG2 300b poll AS1 100 for alarm messages that are possible (3a, 3b). AG1 300a receives (4) and confirms (5) A1. AG2 300b does not receive any alarm message assigned to AG2 300b. At the same time, the following actions are performed now: AG1 300a triggers the alarm signal for A1 at the user B1 (6), AS1 100 starts the timer T5 (7) for monitoring the acknowledgment by the user to AG1 300a, and AS1 100 sends a confirmation of receipt relative to the alarm communication from AG1 300a to AQ1 200 (8). With the receipt of the confirmation from AG1 300a that the alarm was triggered at the user, the timers T3 and T4 (see below) are started. The case in which an alarm signal was triggered by an alarm generator 300 but this was not acknowledged by the user is secured via T3. If no additional alarm generator (e.g., AG2) is available in the system 400, the alarm signal would be triggered at AQ1 200 after the expiration of T3.

However, the user has not acknowledged here the alarm at AG1 300a, so that AG1 300a does not communicate any confirmation by a user to AS1 100, and AS1 100 therefore also fails to forward a confirmation by a user to AQ1 200. Therefore, the mentioned “alarm communication” to AG2 300b now takes place according to the pull method according to the present invention. Not only is the receipt confirmed in this example by AG2 300b to AS1 100 and thus also to AQ1 200, but a user is also confirmed from AG2 300b to AS1 100 and then also to AQ1 200.

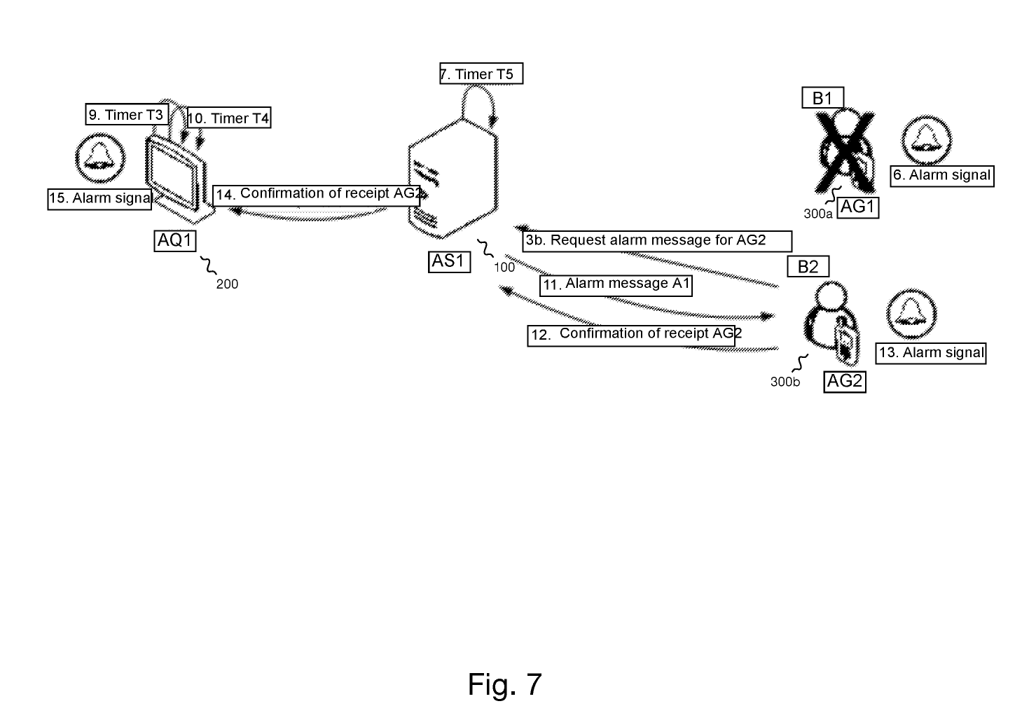

FIG. 7 shows an additional exemplary embodiment of an alarm system 400 with absence of acknowledgment by a user at the alarm source 200. AG2 300b receives the alarm A1 in this exemplary embodiment, but the user B2 does not acknowledge the alarm at the alarm source 200. If an alarm signal was triggered by an alarm generator 300a, but it was not acknowledged by a user before the expiration of T5, the alarm message is released for all alarm generators 300a, 300b: AG2 300b sends the confirmation of receipt (12) and triggers the alarm signal (13). The confirmation of receipt is forwarded from AS1 100 to AQ1 200 (14). Unless the alarm was acknowledged at AQ1 200 or the alarm condition was revoked before the expiration of T4 at the latest, the alarm signal is triggered at AQ1 200 (15). The control module 26 of the alarm source may be configured in some exemplary embodiments to adapt at least one (also a plurality of or all) the predefined times T1, T2, T3, T4 according to the priority of the alarm.

In another exemplary embodiment, AQ1 200 detects the alarm condition and an alarm is sent to B1 via AG1 300a. B1 at first receives the alarm, but he then gives it back. Analogously to the last scenario, an alarm message can be forwarded by the alarm server 100 from an alarm generator 300a already generating an alarm to another one not only when there is no confirmation by the user, but also when the user actively rejects an alarm signal. This may happen both before and also after an acknowledgment by the user. A rejection message is sent in this case to the alarm server 100, so that it is not necessary to wait for the expiration of T5 until the alarm message is forwarded.

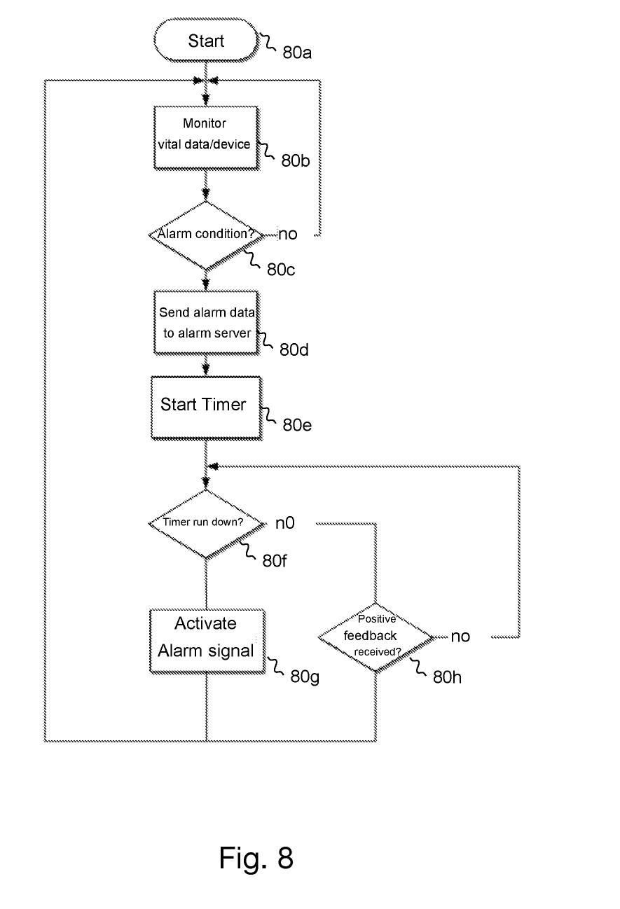

FIG. 8 shows a block diagram of a flow chart of a process for an alarm source in an exemplary embodiment. The flow chart shows the patient monitoring at the alarm source, the communication with the alarm server and the local alarm generation. The process begins with step 80a. The alarm source analyzes in an alarm source monitoring function all alarm conditions from 80b and monitors the vital data or the status of a medical device. If the alarm condition is met 80c, a message with all the alarm information (e.g., identification of the alarm, identification of the alarm source, if necessary, also measured values and alarm limits, etc.) is communicated to the alarm server, 80d, and the process waits for a confirmation of receipt. If no alarm condition exists, the process returns to step 80b. A timer is started, 80e, by means of which the alarm generation is monitored at the alarm generator. If no confirmation 80h (positive feedback) is detected at the alarm source until the rundown of the timer, 80f, the alarm signal is triggered at the alarm source, 80g. Different timers may preferably be used for all individual confirmations, namely,

confirmation of the alarm message by the alarm server,

confirmation of the alarm message by the alarm generator,

confirmation of the alarm signal at the alarm generator by the user, and

confirmation of the alarm signal at the alarm source by the user,
in order to generate an alarm directly at the alarm source without loss of time in case of failure of a component or the absence of an action of the user.

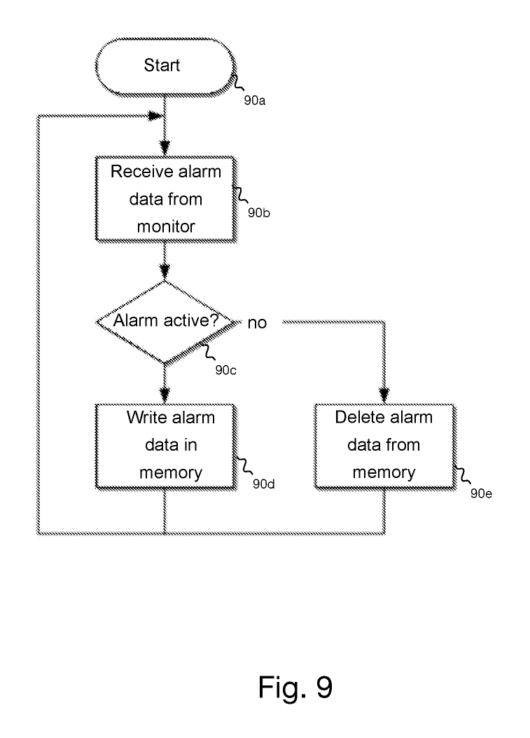

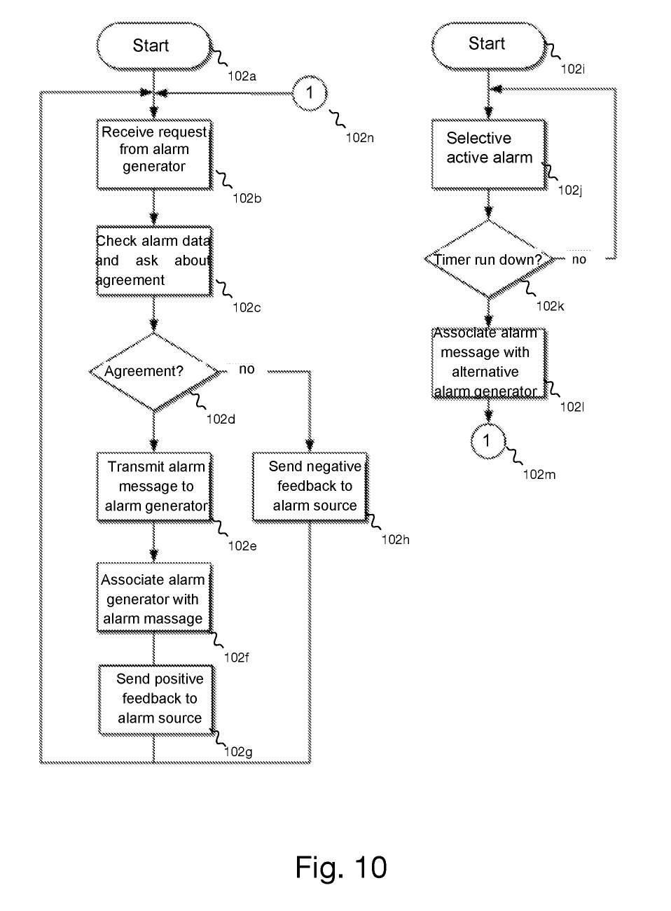

FIG. 9 shows a block diagram of a flow chart of a process for an alarm server in an exemplary embodiment. FIG. 10 shows a block diagram of a flow chart of a process for an alarm server and an alarm generator in an exemplary embodiment. The communication on the alarm server is distributed between two mutually independent activities, namely a) the processing of the alarm messages sent from the alarm sources, cf. FIG. 9, and b) the processing of the alarm requests sent by the alarm generators, cf. FIG. 10. FIG. 9 shows communication with the alarm source. The process begins in step 90a. The alarm server receives the alarm messages of the alarm sources, 90b. The alarm status obtained indicates in this case whether an alarm signal shall be triggered or revoked, 90c. Depending on the alarm status, the corresponding alarm message is stored in the memory together with the identification of the alarm source, 90d, or deleted from the memory, 90e.

FIG. 10 shows the communication with the alarm generator. The process begins with step 102a. As is shown on the left-hand side of FIG. 10, the alarm server receives the alarm requests from the alarm generators, 102b. The requests may also contain characteristics of the user and of the alarm generator, in addition to the identification of the particular alarm generator. Identical alarm messages are searched for in the memory on the basis of these characteristics, 102c. The alarm message with the closest agreement is selected, 102d. The alarm message is sent to the corresponding alarm generator, 102e, and associated with the alarm generator, 102f. Furthermore, a confirmation message on the successful alarm assignment is sent to the alarm source, 102g. Should no suitable alarm messenger be available (no alarm generator collects the information on the alarm), a negative feedback is sent, 102h. To check whether an alarm signal is acknowledged by the user (see diagram on the right-hand side, start with step 102i), all timeouts of the active alarms are checked. The active alarms are selected for this, 102j. If a time has expired, 102k, an alternative alarm generator is identified, and is associated with the alarm message for collecting, 102l. As long as the timer has not run down, the process branches back to step 102k. The procedure on the left-hand side can then correspondingly be run, which is illustrated in FIG. 10 by the two steps “l” 102m, 102n.

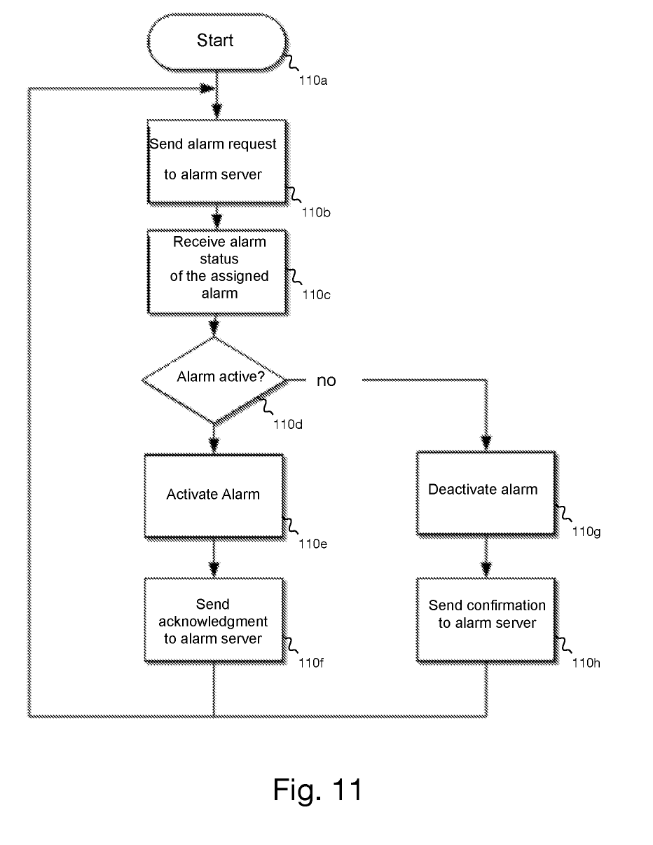

FIG. 11 shows a block diagram of a process for an alarm generator in an exemplary embodiment. FIG. 11 shows the communication with the alarm server and the alarm generation. The process begins in step 110a. The alarm generator sends alarm requests to the alarm server 110b. The alarm requests may be expanded by additional user characteristics, which contain, for example, the user identification, the current position or also core duties of the user. Upon receipt of the assigned alarm, 110c, a new alarm is triggered, 110e, or revoked, 110g, depending on the alarm status, 110d. A confirmation of this is subsequently sent back to the alarm server, 110h. If a new alarm was triggered, 110e, the user can acknowledge this at the alarm generator, 110f. The confirmation of the acknowledgment is likewise reported back to the alarm server, so that forwarding of the alarm to another alarm generator due to the rundown of the acknowledgment timer (T5) is avoided in this case.

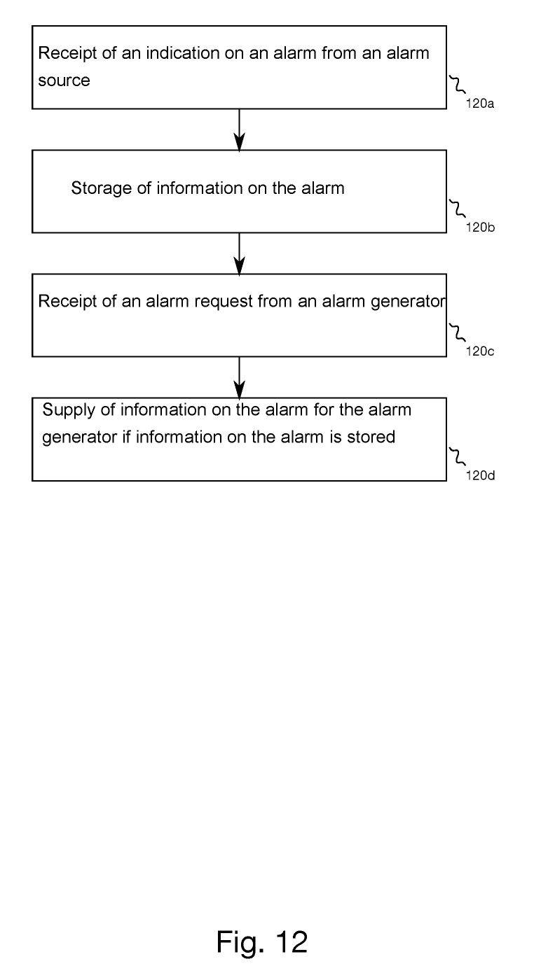

FIG. 12 shows a block diagram of a flow chart of a process for an alarm server in another exemplary embodiment. The process for the alarm server 100 comprises the receipt, 120a, of an indication of an alarm from an alarm source 200 as well as a storage, 120b, of information on the alarm. The process further comprises the receipt, 120c, of an alarm request from an alarm generator 300 and the supply, 120d, of information on the alarm to the alarm generator 300 if information on the alarm is stored.

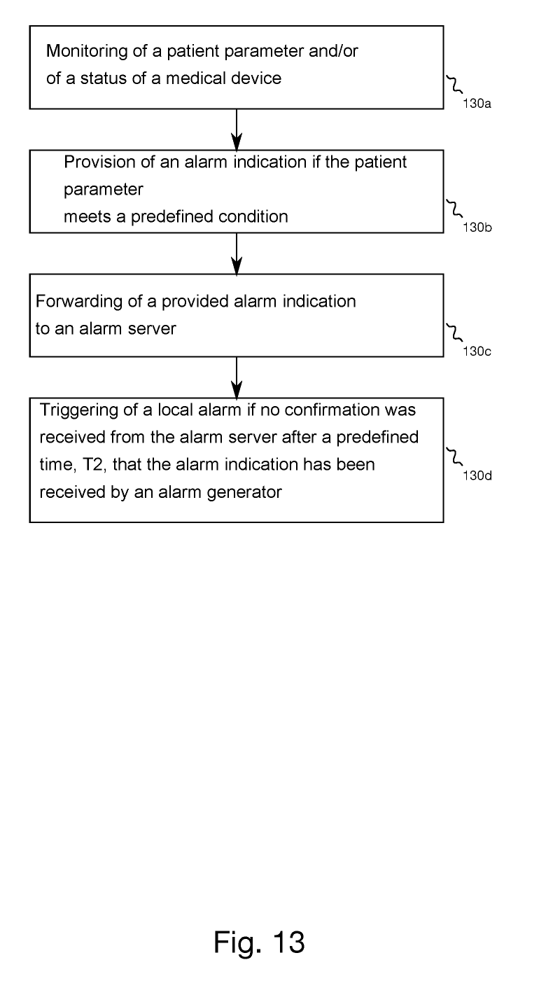

FIG. 13 shows a block diagram of a flow chart of a process for an alarm source in another exemplary embodiment. The process for the alarm source 200 comprises the monitoring, 130a, of a patient parameter and/or of a status of a medical device as well as the supply, 130b, of an alarm indication if the patient parameter meets a predefined condition. The process comprises the forwarding, 130c, of the alarm indication provided to an alarm server 100 and the triggering, 130d, of a local alarm if no confirmation was received from the alarm server 100 after a predefined time, T2, that the alarm indication has been received by an alarm generator 300.

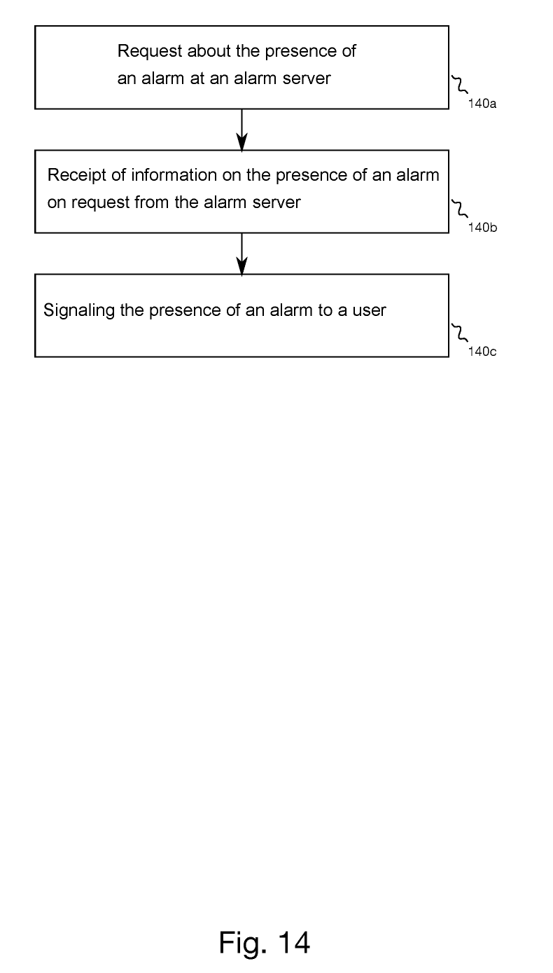

FIG. 14 shows a block diagram of a flow chart of a process for an alarm generator in another exemplary embodiment. The process for the alarm generator 300 comprises a request 140a for the presence of an alarm at an alarm server 100. The process further comprises the receipt, 140b, of information on the presence of an alarm on request from the alarm server 100 and signaling, 140c, of the presence of an alarm to a user.

Another exemplary embodiment is a program with a program code for executing one of the processes being described here, if the program code is executed on a computer, a processor or a programmable hardware component.

The features disclosed in the above description, in the claims and in the drawings may be significant both individually and in any combination for the implementation of exemplary embodiments in the different configurations thereof and, unless something else appears from the description, they may be combined with one another as desired.

Even though some aspects were described in connection with a device, it is obvious that these aspects also represent a description of the corresponding process, so that a block or a component of a device shall also be defined as a corresponding process step or as a feature of a process step. Analogously to this, aspects that were described in connection with or as a process step also represent a description of a corresponding block or detail or feature of a corresponding device.

Depending on certain implementation requirements, exemplary embodiments of the present invention may be implemented in hardware or in software. The implementation may be carried out with the use of a digital storage medium, for example, a floppy disk, a DVD, a Blu-Ray disk, a CD, a ROM, a PROM, an EPROM, an EEPROM or a FLASH memory, a hard drive or another magnetic or optical memory, on which electronically readable control signals are stored, which can or do interact with a programmable hardware component such that the respective process is executed.

A programmable hardware component may be formed by one or more processors, a computer processor (CPU=Central Processing Unit), a graphics processor (GPU=Graphics Processing Unit), a computer, a computer system, an application-specific integrated circuit (ASIC=Application-Specific Integrated Circuit), an integrated circuit (IC=Integrated Circuit), a system on chip (SPC=System on Chip), a programmable logic element or a field-programmable gate array with a microprocessor (FPGA=Field Programmable Gate Array).

The digital storage medium may therefore be machine- or computer-readable. Some exemplary embodiments consequently comprise a data storage medium, which has electronically readable control signals, which are capable of interacting with a programmable computer system or with a programmable hardware component such that one of the processes being described here is executed. An exemplary embodiment is consequently a data storage medium (or a digital storage medium or a computer-readable medium), on which the program for executing one of the processes being described here is recorded.

Exemplary embodiments of the present invention may generally be implemented as a program, firmware, computer program or computer program product with a program code or as data, wherein the program code or the data acts/act such as to execute one of the processes when the program runs on a processor or a programmable hardware component. The program code or the data may also be stored, for example, on a machine-readable carrier or data storage medium. The program code or the data may be present, among other things, as source code, machine code or byte code as well as other intermediate code.

Another exemplary embodiment is, further, a data stream, a signal sequence or a sequence of signals, which data stream or signal sequence represent the program for executing one of the processes being described here. The data stream, the signal sequence or the sequence of signals may be configured, for example, such as to be transferred via a data communication connection, for example, over the Internet or another network. Exemplary embodiments are thus also signal sequences representing data, which are suitable for transmission over a network or a data communication link, wherein the data represent the program.

A program according to an exemplary embodiment may implement one of the processes during the execution thereof by reading storage locations or writing a datum or a plurality of data into these locations, whereby switching processes or other processes are elicited in transistor structures, in amplifier structures or in other electric, optical, magnetic components or components operating according to another principle of function. Data, values, sensor values or other information can correspondingly be detected, determined or measured by a program by reading a storage location. A program can therefore detect, determine or measure variables, values, measured variables and other information by reading one or more storage locations as well as bring about, prompt or execute an action by writing into one or more storage locations as well as actuate other devices, machines and components.

The above-described exemplary embodiments represent only an illustration of the principles of the present invention. It is apparent that modifications and variations of the arrangements and details being described will become apparent to other persons skilled in the art. It is therefore intended that the present invention shall be limited only by the scope of protection of the following patent claims rather than by the specific details, which were presented herein on the basis of the description and the explanation of the exemplary embodiments.

While specific embodiments of the invention have been shown and described in detail to illustrate the application of the principles of the invention, it will be understood that the invention may be embodied otherwise without departing from such principles.

What is claimed is:

1. An alarm server device comprising:
one or more interfaces for communication with one or more alarm sources and with one or more alarm generators;
a memory for storing information relating to one or more alarms; and
a control module configured to control the one or more interfaces and the memory, configured to receive an indication relating to an alarm via the one or more interfaces from an alarm source and to store the information relating to the alarm in the memory and configured to receive an alarm request from an alarm generator via the one or more interfaces and to provide the alarm for the alarm generator upon receipt of the alarm request if information relating to the alarm is stored in the memory.

2. An alarm server device in accordance with claim 1, wherein:
the indication relating to an alarm comprises an indication of an alarm source that indicates a state of an alarm of the alarm source; and
the control module is configured to store status information relating to a state of alarm of the alarm source in the memory.

3. An alarm server device in accordance with claim 1, wherein the control module is configured to provide or confirm the information relating to the alarm to the alarm generator according to a pull method for the alarm generator.

4. An alarm server device in accordance with claim 1, wherein the control module is configured to request the indication relating to the alarm at the alarm source according to a pull method for the alarm source or to receive the indication relating to the alarm according to a push method for the alarm source or both to request the indication relating to the alarm at the alarm source according to a pull method for the alarm source and also to receive the indication relating to the alarm according to a push method for the alarm source.

5. An alarm server device in accordance with claim 1, wherein the control module is configured to store one or more components of a group comprising: an identification of the alarm source; properties of the alarm; a category of the alarm; a location of the alarm; a priority of the alarm in the memory associatively with the indication, and to forward it to the alarm generator (300) with confirmation of the alarm.

6. An alarm server device in accordance with claim 1, wherein the control module is configured to trigger a forwarding to additional alarm generators or to trigger a local alarm at the alarm server or to trigger both a forwarding to additional alarm generators and a local alarm at the alarm server, after confirmation of the presence of an alarm to the alarm generator if the alarm generator fails to confirm an acknowledgment of a presence of an alarm by a user after a predefined time.

7. An alarm source device comprising:
one or more interfaces for communication with an alarm server;
a monitoring device for monitoring a patient parameter or monitoring a status of a medical device or monitoring both a patient parameter and a status of a medical device and for providing an alarm indication if the patient parameter or status meets a predefined condition; and
a control module configured to control the one or more interfaces and the monitoring device and configured to forward an alarm indication provided by the monitoring device via the one or more interfaces to the alarm server and configured to trigger a local alarm at the alarm source after receipt of an alarm indication from the monitoring device if no confirmation was received from the alarm server after a predefined time that the alarm indication has been received by an alarm generator.

8. An alarm source device in accordance with claim 7, wherein the monitoring device is configured to monitor the patient parameter taking into account at least one sensor signal of a medical device or taking into account at least one operating parameter of an actuator of a medical device relative to the predefined condition or taking into account at least one sensor signal of a medical device and at least one operating parameter of an actuator of a medical device relative to the predefined condition.

9. An alarm source device in accordance with claim 7, wherein:
the control module is configured to provide the alarm indication via an alarm for the alarm server upon request by the alarm server according to a pull method for the alarm source; or
to provide the alarm indication relating to the alarm to the alarm server according to a push method for the alarm source; or
the control module is configured to provide the alarm indication via an alarm for the alarm server upon request by the alarm server according to a pull method for the alarm source and to provide the alarm indication relating to the alarm to the alarm server according to a push method for the alarm source.

10. An alarm source device in accordance with claim 7, wherein
the predefined time corresponds to a second predefined time;
the control module is configured to trigger a local alarm at the alarm source upon receipt of an alarm indication from the monitoring device if no confirmation was received from the alarm server after another predefined time, corresponding to a first predefined time, that the alarm indication has been received by the alarm server.

11. An alarm source device in accordance with claim 10, wherein the control module is configured to trigger a local alarm at the alarm source upon receipt of an alarm indication from the monitoring device if no confirmation was received from the alarm server after a third predefined time that the alarm indication has been acknowledged by a user.

12. An alarm source device in accordance with claim 11, wherein the control module is configured to trigger a local alarm at the alarm source upon receipt of an alarm indication from the monitoring device if no acknowledgment of the alarm indication by a user has been received at the alarm source after a fourth predefined time and the patient parameter still continues to meet the predefined condition.

13. An alarm source device in accordance with claim 12, wherein the control module is configured to adapt at least one of the predefined times according to the priority of the alarm.

14. An alarm generator device comprising:
one or more interfaces for communication with an alarm server;
an output device for outputting information relating to an alarm to a user; and
a control module configured to control the one or more interfaces and the output device and configured to poll an alarm server via the one or more interfaces about a presence of an alarm and to receive information relating to the presence of an alarm at the alarm server on request and configured to signal the presence of the alarm to the user via the output device upon receipt of the information relating to the presence of an alarm from the alarm server.

15. An alarm generator device in accordance with claim 14, wherein the control module is configured to poll the alarm server about a presence of an alarm according to a pull method for the alarm generator device.

16. An alarm generator device in accordance with claim 14, wherein the control module is configured to poll the alarm server about the presence of alarms at regular, configurable time intervals in based on an event or as a function of an operating state of the alarm generator device.

17. An alarm generator device in accordance with claim 14, wherein the control module is configured to trigger a local alarm at the alarm generator device upon receipt of the information or after confirmation of the presence of an alarm from the alarm server if no acknowledgment of the presence of an alarm by a user was obtained after a predefined time.

18. An alarm generator device in accordance with claim 17, wherein the control module is configured to adapt the predefined time according to a priority of the alarm.

19. An alarm system comprising:
alarm server device comprising one or more interfaces for communication with one or more alarm sources and with one or more alarm generators, a memory for storing information relating to one or more alarms, and an alarm server control module configured to control the one or more interfaces and the memory, configured to receive an indication relating to an alarm via the one or more interfaces from an alarm source and to store information relating to the alarm in the memory and configured to receive an alarm request from an alarm generator via the one or more interfaces and to provide the alarm for the alarm generator upon receipt of the alarm request if information relating to the alarm is stored in the memory;
an alarm source device comprising one or more interfaces for communication with the alarm server, a monitoring device for monitoring a patient parameter or monitoring a status of a medical device or monitoring both a patient parameter and a status of a medical device and for providing an alarm indication if the patient parameter or status meets a predefined condition, and an alarm source control module configured to control the one or more interfaces and the monitoring device and configured to forward an alarm indication provided by the monitoring device via the one or more interfaces to the alarm server and configured to trigger a local alarm at the alarm source after receipt of an alarm indication from the monitoring device if no confirmation was received from the alarm server after a predefined time that the alarm indication has been received by an alarm generator; and
the alarm generator device comprising one or more interfaces for communication with the alarm server, an output device for outputting information relating to an alarm to a user, and an alarm generator control module configured to control the one or more interfaces and the output device and configured to poll the alarm server via the one or more interfaces about a presence of an alarm and to receive information relating to the presence of an alarm at the alarm server on request and configured to signal the presence of the alarm to the user via the output device upon receipt of the information relating to the presence of an alarm from the alarm server.

20. An alarm server process comprising the steps of:
providing an alarm server device comprising one or more interfaces for communication with one or more alarm sources and with one or more alarm generators, a memory for storing information relating to one or more alarms, and a control module configured to control the one or more interfaces and the memory, configured to receive an indication relating to an alarm via the one or more interfaces from an alarm source and to store information relating to the alarm in the memory and configured to receive an alarm request from an alarm generator via the one or more interfaces and to provide the alarm for the alarm generator upon receipt of the alarm request if information relating to the alarm is stored in the memory;
receiving an indication of an alarm from an alarm source;
storing information relating to the alarm;
receiving an alarm request from an alarm generator; and
supplying information relating to the alarm to the alarm generator if information relating to the alarm is stored.

21. An alarm server process according to claim 20, further comprising providing a computer program with a program code for executing the steps of receiving an indication of an alarm, storing information relating to the alarm, receiving an alarm request from an alarm generator; and supplying information relating to the alarm to the alarm generator, wherein the program code is executed on a computer, a processor or a programmable hardware component.

22. An alarm source process comprising the steps of:
providing an alarm source device comprising one or more interfaces for communication with an alarm server, a monitoring device for monitoring a patient parameter or monitoring a status of a medical device or monitoring both a patient parameter and a status of a medical device and for providing an alarm indication if the patient parameter or status meets a predefined condition, and a control module configured to control the one or more interfaces and the monitoring device and configured to forward an alarm indication provided by the monitoring device via the one or more interfaces to the alarm server and configured to trigger a local alarm at the alarm source after receipt of an alarm indication from the monitoring device if no confirmation was received from the alarm server after a predefined time that the alarm indication has been received by an alarm generator;
monitoring a patient parameter or monitoring a status of a medical device or monitoring a patient parameter and a status of a medical device;
providing an alarm indication if the patient parameter or medical device status meets a predefined condition;
forwarding a provided alarm indication to an alarm server; and
triggering a local alarm if no confirmation of the alarm server was received after a predefined time that the alarm indication has been received by an alarm generator.

23. An alarm source process according to claim 22, further comprising providing a computer program with a program code for executing the steps of monitoring a patient parameter or monitoring a status of a medical device or monitoring a patient parameter and a status of a medical device, providing an alarm indication, forwarding a provided alarm indication and triggering a local alarm, wherein the program code is executed on a computer, a processor or a programmable hardware component.

24. An alarm generator process comprising:
providing an alarm generator device comprising one or more interfaces for communication with an alarm server, an output device for outputting information relating to an alarm to a user, and a control module configured to control the one or more interfaces and the output device and configured to poll an alarm server via the one or more interfaces about a presence of an alarm and to receive information relating to the presence of an alarm at the alarm server on request and configured to signal the presence of the alarm to the user via the output device upon receipt of the information relating to the presence of an alarm from the alarm server;
polling for a presence of an alarm at an alarm server;
receiving information relating to a presence of an alarm on request from the alarm server; and
signaling a presence of an alarm at a user.

25. An alarm generator process according to claim 24, further comprising providing a computer program with a program code for executing the steps of polling for a presence of an alarm at an alarm server, receiving information relating to a presence of an alarm, and signaling a presence of an alarm at a user, wherein the program code is executed on a computer, a processor or a programmable hardware component.
